(19) United States
(12) Patent Application Publication
(10) Pub. No.: US 2025/0339067 A1
(43) Pub. Date: Nov. 6, 2025

(54) Line Draw Device

(71) Applicant: Becton, Dickinson and Company, Franklin Lakes, NJ (US)

(72) Inventors: Jonathan Karl Burkholz, Salt Lake City, UT (US); Curtis H. Blanchard, Herriman, UT (US); Erica E. Neumann, Sandy, UT (US); Weston F. Harding, Lehi, UT (US)

(21) Appl. No.: 19/192,482

(22) Filed: Apr. 29, 2025

Related U.S. Application Data
(60) Provisional application No. 63/641,599, filed on May 2, 2024.

(51) Int. Cl. A61B 5/15; A61M 25/00; A61M 39/02; A61M 39/10

(52) U.S. Cl. CPC A61B 5/150992; A61M 39/10; A61M 2025/0004; A61M 25/0097; A61M 2039/0202; A61M 2039/0258; A61M 2039/0276; A61M 2205/0216

(57) ABSTRACT

A line draw device for use with a peripheral intravenous catheter (PIVC) includes a housing and a flow tube, the flow tube having a retracted position where the flow tube is received within the housing and an extended position where the flow tube extends beyond the housing, a support tube connected to the flow tube and moveable with the flow tube, and a collapsible support positioned within the housing, with a portion of the flow tube extending through the collapsible support. The collapsible support has a first length when the flow tube is in the retracted position and a second length when the flow tube is in the extended position, with the first length larger than the second length. A distal end of the support tube is configured to be positioned proximally of a needlefree connector when the line draw device is connected thereto.

### CROSS-REFERENCE TO RELATED APPLICATION

The present application claims priority to U.S. Provisional Patent Application No. 63/641,599 entitled “Line Draw Device” filed May 2, 2024, the entire disclosure of which is hereby incorporated by reference.

### BACKGROUND OF THE INVENTION

### Field of the Invention

The present disclosure relates generally to line draw devices and, more specifically, to line draw devices with various features for supporting a flow tube of the line draw device and ensuring a secure connection between the line draw device and other components.

### Description of Related Art

Line draw devices, when used with indwelling IV catheters, can include displaceable flow tubes or catheters that are advanced beyond the tip of the indwelling catheter for blood collection. Often, when the displaceable catheter is advanced, it can encounter an obstruction, resulting in deflection of the catheter. Examples of obstructions include the friction of the seal within the line draw device, torturous path within an integrated catheter, pinching of the catheter tubing as it dives into the skin, thrombus, fibrin, and valves. Such deflections can reduce the ability of the displaceable catheter to extend beyond the tip of the indwelling catheter, and thus limits their use for blood collection. Some line draw devices include a support tube along a portion of the displaceable catheter to prevent buckling of the displaceable catheter during use.

### SUMMARY OF THE INVENTION

In accordance with an embodiment of the present invention, a line draw device for use with a peripheral intravenous catheter (PIVC) includes a housing having a proximal end, a distal end, and a sidewall therebetween defining an inner volume, a flow tube defining a lumen, the flow tube having a retracted position where a distal end of the flow tube is received within the housing and an extended position where the distal end of the flow tube extends beyond the distal end of the housing, a support tube connected to the flow tube and moveable with the flow tube, and a collapsible support positioned within the housing, with a portion of the flow tube extending through the collapsible support. The collapsible support has a first length when the flow tube is in the retracted position and a second length when the flow tube is in the extended position, with the first length larger than the second length. A distal end of the support tube is configured to be positioned proximally of a distal end of a needlefree connector when the line draw device is connected to the needlefree connector.

In certain embodiments, the support tube includes an accordion fold. In other configurations, the support tube includes telescoping segments.

In accordance with another embodiment of the present invention, a line draw device for use with a peripheral intravenous catheter (PIVC) includes a housing having a proximal end, a distal end, and a sidewall therebetween defining an inner volume, a hub received within the housing, a flow tube connected to the hub and defining a lumen, the flow tube having a retracted position where a distal end of the flow tube is received within the housing and an extended position where the distal end of the flow tube extends beyond the distal end of the housing, and a lock member, the lock member configured to engage the hub or the housing when the flow tube is in the extended position.

In accordance with another embodiment of the present invention, a line draw device for use with a peripheral intravenous catheter (PIVC) includes a housing having a proximal end, a distal end, and a sidewall therebetween defining an inner volume, a flow tube defining a lumen, the flow tube having a retracted position where a distal end of the flow tube is received within the housing and an extended position where the distal end of the flow tube extends beyond the distal end of the housing, and a support tube connected to the flow tube and moveable with the flow tube when the flow tube moves between the retracted position and the extended position, wherein the support tube is flexible.

In certain configurations, the support tube includes polyimide, polyurethane, Nitinol, or semi-rigid material more flexible than steel. In other configurations, the support tube includes a steel wire coil. In certain configurations, the housing includes a connector at the distal end, with the connector configured to be connected to a needlefree connector or indwelling catheter.

In accordance with another embodiment of the present invention, a line draw device for use with a peripheral intravenous catheter (PIVC) includes a housing having a proximal end, a distal end, and a sidewall therebetween defining an inner volume, a flow tube defining a lumen, the flow tube having a retracted position where a distal end of the flow tube is received within the housing and an extended position where the distal end of the flow tube extends beyond the distal end of the housing, and a support tube connected to the flow tube and moveable with the flow tube when the flow tube moves between the retracted position and the extended position, wherein a transition between the support tube and the flow tube is configured to reduce damage to the PIVC, related components, or the line draw device.

In certain configurations, the transition between the support tube and the flow tube includes an overmold, a tapered segment, a chamfer, and/or radius. In other configurations, the transition between the support tube and the flow tube includes an asymmetrical tip of the support tube.

In accordance with another embodiment of the present invention, a line draw device for use with a peripheral intravenous catheter (PIVC) includes a housing having a proximal end, a distal end, and a sidewall therebetween defining an inner volume, a flow tube defining a lumen, the flow tube having a retracted position where a distal end of the flow tube is received within the housing and an extended position where the distal end of the flow tube extends beyond the distal end of the housing, and a support tube connected to the flow tube and moveable with the flow tube when the flow tube moves between the retracted position and the extended position, wherein the housing is flexible.

In certain configurations, the housing includes one or more flexible sections.

In accordance with another embodiment of the present invention, a line draw device for use with a peripheral intravenous catheter (PIVC) includes a housing having a proximal end, a distal end, and a sidewall therebetween defining an inner volume, a hub received within the housing, a flow tube connected to the hub and defining a lumen, the flow tube having a retracted position where a distal end of the flow tube is received within the housing and an extended position where the distal end of the flow tube extends beyond the distal end of the housing, an actuator connected to the hub, at least a portion of the actuator extends from the proximal end of the housing, and an extension tube connected to the actuator or the hub.

In accordance with another embodiment of the present invention, a line draw device for use with a peripheral intravenous catheter (PIVC) includes a housing having a proximal end, a distal end, and a sidewall therebetween defining an inner volume, a hub received within the housing, a flow tube connected to the hub and defining a lumen, the flow tube having a retracted position where a distal end of the flow tube is received within the housing and an extended position where the distal end of the flow tube extends beyond the distal end of the housing, and a connector positioned at the distal end of the housing, the connector comprising a male luer lock connector and a moveable lock member, wherein, when a corresponding component is received by the male luer lock connector, the moveable lock member configured to engage the corresponding component to lock the corresponding component to the housing.

In accordance with another embodiment of the present invention, a line draw device for use with a peripheral intravenous catheter (PIVC) includes a housing having a proximal end, a distal end, and a sidewall therebetween defining an inner volume, a connector positioned at the distal end of the housing, a flow tube defining a lumen, the flow tube having a retracted position where a distal end of the flow tube is received within the housing and an extended position where the distal end of the flow tube extends beyond the distal end of the housing, and a support tube connected to the flow tube and moveable with the flow tube when the flow tube moves between the retracted position and the extended position, wherein a distal end of the support tube is positioned proximally of a distal end of the connector when the flow tube is in the extended position and when a distal end of the flow tube has a maximum extension from the distal end of the connector.

In accordance with another embodiment of the present invention, a system includes an integrated catheter having a catheter adapter with a side port and needlefree connector in fluid communication with the side port, and a line draw device. The line draw device includes a housing having a proximal end, a distal end, and a sidewall therebetween defining an inner volume, a connector positioned at the distal end of the housing, with the connector configured to be connected to the needlefree connector, a flow tube defining a lumen, with the flow tube having a retracted position where a distal end of the flow tube is received within the housing and an extended position where the distal end of the flow tube extends beyond the distal end of the housing, and a support tube connected to the flow tube and moveable with the flow tube when the flow tube moves between the retracted position and the extended position. A distal end of the support tube is positioned proximally of an internal wall of the catheter adapter when the flow tube is in the extended position and when a distal end of the flow tube has a maximum extension from the distal end of the connector.

In certain configurations, the distal end of the support tube is positioned at a centerline of the catheter adapter when the flow tube is in the extended position and when the distal end of the flow tube has a maximum extension from the distal end of the connector.

In accordance with yet another embodiment of the present invention, a line draw device for use with a peripheral intravenous catheter (PIVC) includes a housing having a proximal end, a distal end, and a sidewall therebetween defining an inner volume, the housing defining a track, a hub received within the housing, the hub comprising a guide received within the track of the housing, and a flow tube connected to the hub and defining a lumen, the flow tube having a retracted position where a distal end of the flow tube is received within the housing and an extended position where the distal end of the flow tube extends beyond the distal end of the housing, wherein the track comprises a retraction inhibition feature.

In certain configurations, the retraction inhibition feature includes track structure configured to increase a force required to move the flow tube from the extended position to the retracted position. In other configurations, the retraction inhibition feature include non-linear track portions.

### BRIEF DESCRIPTION OF THE DRAWINGS

FIG. 1 is a front view of a conventional line draw device;

FIG. 2 is a schematic illustration of a conventional flow tube and support tube arrangement;

FIG. 3 is a schematic illustration of a conventional line draw device in use with an integrated catheter;

FIG. 4 is a schematic illustration of a conventional line draw device in use with an integrated catheter;

FIG. 5 is a schematic illustration of a conventional line draw device in use with an integrated catheter;

FIG. 6 is a schematic illustration of a conventional line draw device in use with an integrated catheter;

FIG. 7 is a schematic illustration of a line draw device in use with an integrated catheter in accordance with an aspect or embodiment of the present application;

FIG. 8 is a schematic illustration of a line draw device in accordance with an aspect or embodiment of the present application, showing an extended position of a support tube;

FIG. 9 is a schematic illustration of the line draw device of FIG. 8, showing a collapsed position of a support tube;

FIG. 10 is a schematic illustration of a line draw device in accordance with an aspect or embodiment of the present application;

FIG. 11 is a schematic illustration of a line draw device in accordance with an aspect or embodiment of the present application, showing a retracted position of a support tube;

FIG. 12 is a schematic illustration of the line draw device of FIG. 11, showing an advanced position of a support tube;

FIG. 13 is a perspective view of a line draw device in accordance with an aspect or embodiment of the present application;

FIG. 14 is a schematic illustration of a line draw device in accordance with an aspect or embodiment of the present application;

FIG. 15 is a schematic illustration of a flow tube and support tube arrangement in accordance with an aspect or embodiment of the present application;

FIG. 16 is a schematic illustration of a flow tube and support tube arrangement in accordance with an aspect or embodiment of the present application;

FIG. 17 is a schematic illustration of a flow tube and support tube arrangement in accordance with an aspect or embodiment of the present application;

FIG. 18 is a schematic illustration of a flow tube and support tube arrangement in accordance with an aspect or embodiment of the present application;

FIG. 19 is a schematic illustration of a flow tube and support tube arrangement in accordance with an aspect or embodiment of the present application;

FIG. 20 is a schematic illustration of a flow tube and support tube arrangement in accordance with an aspect or embodiment of the present application;

FIG. 21 is a schematic illustration of a line draw device in accordance with an aspect or embodiment of the present application;

FIG. 22 is a schematic illustration of a line draw device in accordance with an aspect or embodiment of the present application;

FIG. 23 is a schematic illustration of a line draw device in accordance with an aspect or embodiment of the present application;

FIG. 24 is a schematic illustration of a line draw device in accordance with an aspect or embodiment of the present application;

FIG. 25 is a schematic illustration of a line draw device in accordance with an aspect or embodiment of the present application;

FIG. 26 is a schematic illustration of a line draw device in accordance with an aspect or embodiment of the present application;

FIG. 27 is a schematic illustration of connector of a line draw device in accordance with an aspect or embodiment of the present application;

FIG. 28 is a partial cross-sectional view of connector of a line draw device in accordance with an aspect or embodiment of the present application;

FIG. 29 is a partial cross-sectional view of the connector of FIG. 28, showing insertion of a corresponding component;

FIG. 30 is a perspective view of the connector of FIG. 28, showing insertion of a corresponding component;

FIG. 31 is a schematic illustration of a line draw device in accordance with an aspect or embodiment of the present application;

FIG. 32 is a schematic illustration of a line draw device in accordance with an aspect or embodiment of the present application; and

FIG. 33 is a schematic illustration of a line draw device in accordance with an aspect or embodiment of the present application.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

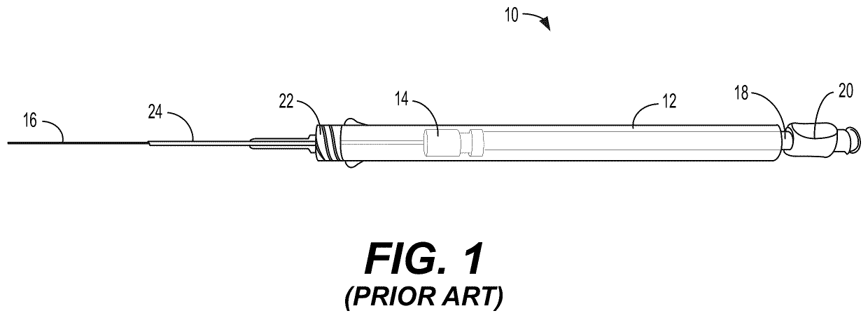

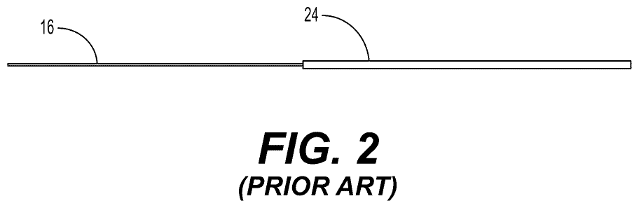

Referring to FIGS. 1 and 2, a conventional line draw device 10 includes a housing 12, a hub 14 received within the housing 12, a flow tube 16 defining a lumen connected to and extending distally from the hub 14, an advancement tube 18 connected to and extending proximally from the hub 14, an advancement handle 20 at a proximal end of the advancement tube 18, the housing 12 includes a connector 22 at a distal end of the housing 12 for connecting the device 10 to an indwelling peripheral intravenous catheter (PIVC) directly or via an intermediate device or connector, and a support tube 24 connected to the flow tube 16 and moveable with the flow tube 16. The connector 22 may be formed integrally or separately from the housing 12. The flow tube 16 is moveable between a retracted position where a distal end of the flow tube 16 is received within the housing 12 and an extended position where the distal end of the flow tube 16 extends beyond the distal end of the housing 12. The line draw device 10 is configured to couple to and/or otherwise engage an indwelling peripheral intravenous catheter (PIVC) to transfer fluid from (e.g., aspiration of blood) and/or transfer fluid to (e.g., infusion of a drug or substance) a portion of a patient. The line draw device 10 may be one of the line draw devices shown and described in U.S. Pat. No. 11,389,624 and U.S. Patent Application Publication No. 2023/0181071, which are hereby incorporated by reference in their entirety.

One or more problems may arise during use of the line draw device 10, including: 1) damage to the line draw device and/or the indwelling catheter as the support tube (typically a metal tube) is advanced into the indwelling catheter or component; 2) loss of flexibility of the indwelling catheter as the support tube is advancement or inadequate flexibility of the system or components, which can result in suboptimal drawing technique with inverted collection tubes that can inhibit proper mixing of the blood with additives in the collection tube and inability to visualize fill levels as well as leading to dislodgement of the indwelling catheter or excessive site manipulation of the indwelling catheter; 3) a propensity for the line draw device 10 to disconnect a component of the indwelling catheter or related component during connection of the line draw device 10 to the indwelling catheter or related component; 4) the connector 22 inadvertently disconnecting from a needlefree connector or indwelling catheter; 5) inadequate reach or extension of the flow tube 16 to achieve optimal blood draw success; 6) potential damage from contact of a blunt cannula or other features of the connector 22 with a needlefree connector or indwelling catheter; and 7) propensity of the line draw device 10 to prematurely and unintentionally retract during a line draw procedure.

The support tube 24 is provided to reduce the likelihood of the flow tube 16 buckling during advancement of the flow tube 16 through the indwelling catheter. The flow tube 16 is connected to and extends distally from the support tube 24 and a portion of the flow tube 16 may be positioned inside at least a portion of the support tubing. A discontinuity is created at a transition between the support tube 24 and the flow tube 16, which may lead to risk of the flow tube 16 buckling, kinking, or being damaged or cut at this location given the support tube 24 may be a thin wall metal tube with an end that may easily damage the flow tube 16 or other polymeric system components. These risks and problems are exacerbated if used with a catheter including an integrated extension set and/or fluid path centerline angle changes, such as the Nexiva integrated catheter commercially available from Becton, Dickinson and Company.

Referring again to FIG. 2, the flow tube 16 and support tube 24 arrangement, as noted above, can introduce problems as it is advanced further through a range of vascular access devices and device types. When used with certain vascular access devices or indwelling catheters, the flow tube 16 and support tube 24 arrangement may lead to buckling and kinking of the flow tube 16, permanent bending and deformation of the support tube 24, risk of cutting or separation of a portion of the tube 16 from the tubing assembly, skiving and damage to components of the indwelling catheter or needlefree connector, including any plastic component the support tube 24 contacts and the catheter tubing, puncturing the catheter or extension set tubing and loss of desirable flexibility of flexible members of the catheter system.

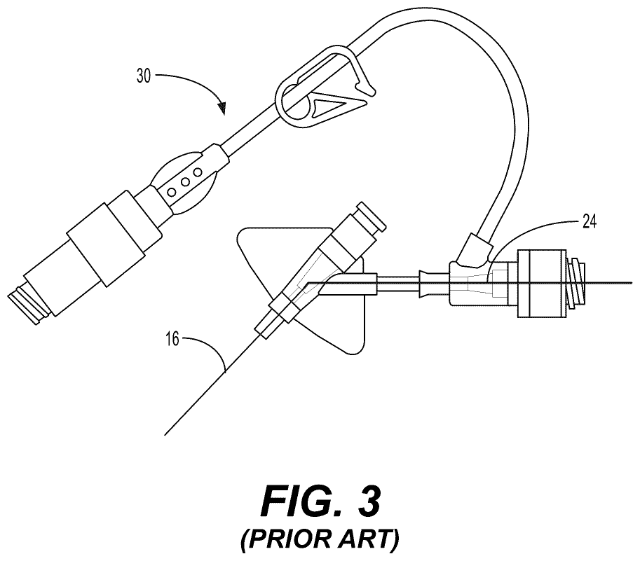

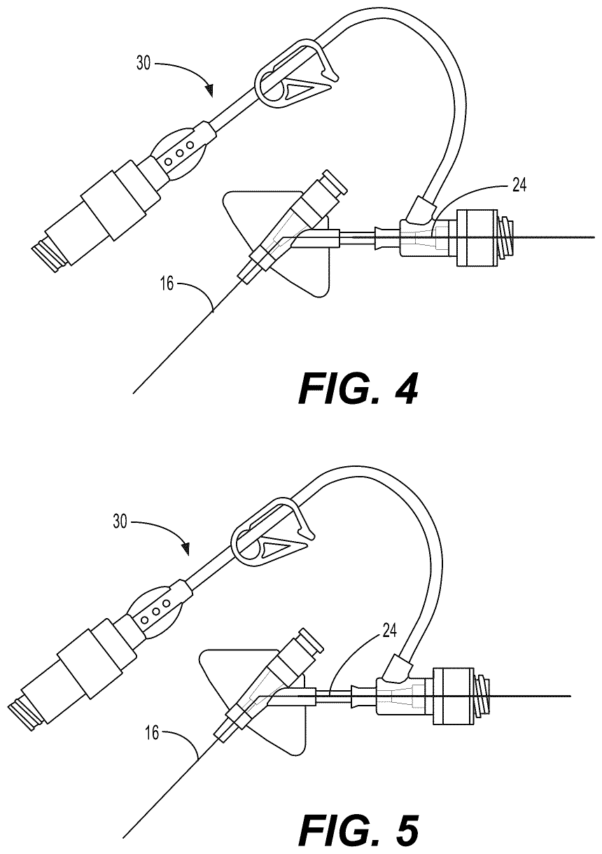

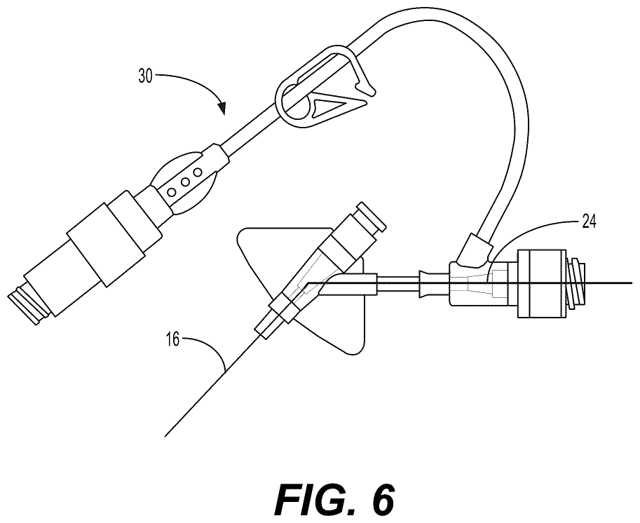

Referring to FIGS. 3-6, schematic illustrations of the line draw device 10 utilized in connection with an integrated catheter 30, such as the Nexiva integrated catheter commercially available from Becton, Dickinson and Company, are shown. As shown in FIG. 3, if the support tube 24 is advanced too far, the support tube 24 can collide with a catheter adapter or internal structure possibly leading to kinking or damage to the flow tube 16. As shown in FIGS. 4 and 5, the support tube 24 can be positioned within a flexible extension tube portion of the integrated catheter 30 or beyond the flexible extension tube portion, which can damage and limit the flexibility of the integrated catheter 30. As shown in FIG. 6, the support tube 24 can damage a catheter adapter or wedge area of the integrated catheter 30, a catheter adapter port ramp, the flexible extension tube portion of the integrated catheter 30, or an inlet into the needlefree connector.

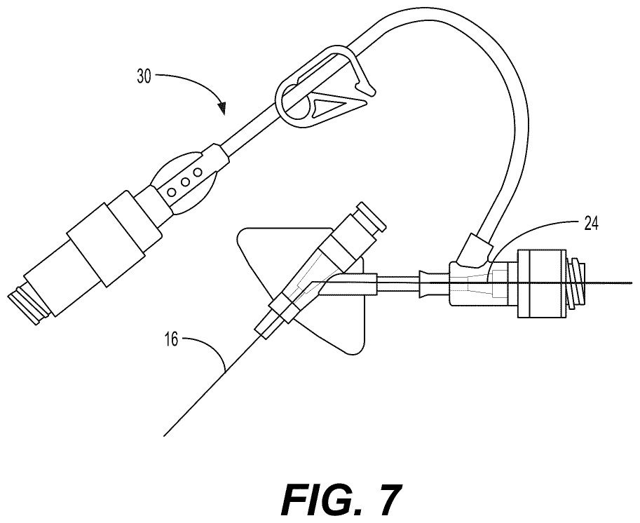

Referring to FIGS. 7-12, according to one aspect or embodiment of the present application, various concepts are shown that include one or more features to ensure a distal edge of the support tube 24 remains proximal to any flexible portion of the fluid path and/or any portion of the fluid path with a centerline and feature angle change that could result in the support tube 24 contacting and damaging the integrated catheter 30 or related components.

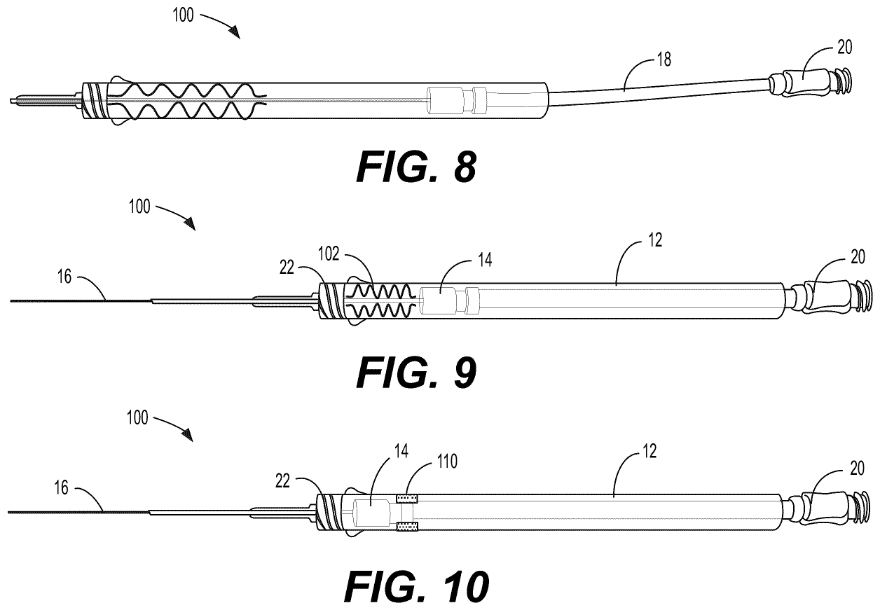

Referring to FIGS. 8-12, according to one aspect or embodiment, a line draw device 100 similar to the line draw device 10 is shown. The line draw device 100, however, includes a collapsible support 102 positioned within the housing 12, a portion of the flow tube 16 extending through the collapsible support 102, with the collapsible support 102 having a first length when the flow tube 16 is in the retracted position and a second length when the flow tube 16 is in the extended position. The first length larger than the second length.

As shown in FIGS. 8 and 9, in one aspect or embodiment, the collapsible support 102 includes an accordion fold to allow the collapsible support 102 to collapse from an expanded state (FIG. 8) to a collapsed state (FIG. 9) as the flow tube 16 and hub 14 are advanced. The collapsible support 102 may formed from an elastomeric material and be configured to collapse due to the material and/or shape of the collapsible support 102. The line draw device 100 includes the support tube 24, as discussed above in connection with the line draw device 10. The support tube 24, however, of the line draw device 100 is shortened such that a distal end of the support tube 24 does not extend past the needlefree connector of the integrated catheter 30. The collapsible support 102 does not move with the flow tube 16 and the support tube 24. In some aspects or embodiments, a distal end of the collapsible support 102 is connected to the housing 12, with a remaining portion of the collapsible support 102 free to move relative to the housing 12. The collapsible support 102 is configured to provide additional support for the flow tube 16 during movement of the flow tube 16 from the retracted position to the extended position. As the advancement handle 20 and/or the advancement tube 18 are moved distally, the flow tube 16 moves relative to the collapsible support 102 while engaging at least a portion of the collapsible support 102 to inhibit kinking or bending during advancement of the flow tube 16 through the integrated catheter 30 and a vein of a patient. In some aspects or embodiments, the collapsible support 102 is continuously in contact with the flow tube 16 or the support tube 24. In some aspects or embodiments, the collapsible support 102 is spaced from the flow tube and only contacts the flow tube 16 during bending of the flow tube 16 from a longitudinal axis of the device 100. The hub 14 engages the collapsible support 102 as the flow tube 16 is transitioned from the retracted position to the extended position to collapse the collapsible support 102, as shown in FIG. 9, which allows for the full extension of the flow tube 16 without adding additional length to the housing 12.

The shortened support tube 24 would allow a portion of the flow tube 16 to be unsupported in the absence of the collapsible support 102. Accordingly, the shortened support tube 24 and the collapsible support 102 allow the flow tube 16 to be supported while ensuring a distal edge of the support tube 24 remains proximal to any flexible portion of the fluid path and/or any portion of the fluid path with a centerline and feature angle change that could result in the support tube 24 contacting and damaging the integrated catheter 30 or related components, as discussed above.

As shown in FIG. 10, in some aspects or embodiments, the line draw device 100 includes a lock member 110 connected to the housing 12, with the lock member 110 configured to engage the hub 14 when the flow tube 16 is in the extended position. In one aspect or embodiment, the lock member 110 is a push button lock or detent that engages one or more sockets or grooves defined by the hub 14 or housing 12. In one aspect or embodiment, the lock member 110 engages a circumferential groove defined by the hub 14. In one aspect or embodiment, the lock member 110 is positioned on the hub 14 and engages the housing 12. The lock member 110 ensures the compressive forces from the collapsible support 102 do not push the flow tube 16 out of the vein during blood collections. In one aspect or embodiment, the lock member 110 is provided by itself without the collapsible support 102 to prevent accidental withdrawal of the flow tube 16.

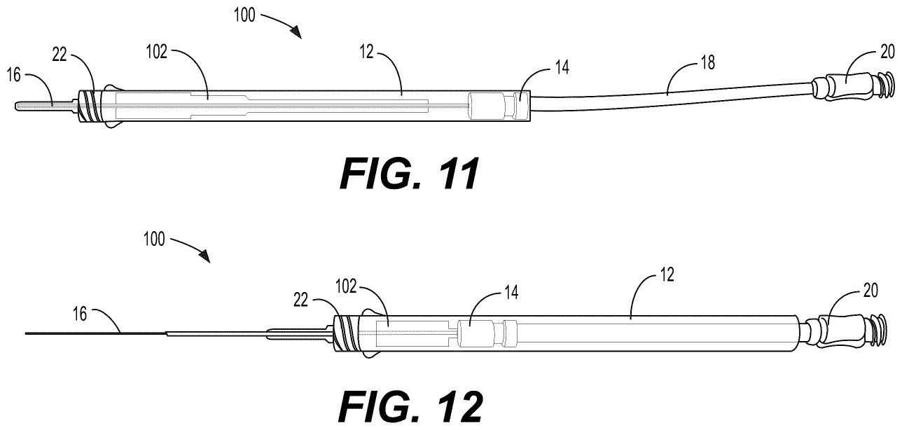

As shown in FIGS. 11 and 12, in one aspect or embodiment, the collapsible support 102 includes telescoping segments to allow the collapsible support 102 to collapse from an expanded state (FIG. 11) to a collapsed state (FIG. 12) as the flow tube 16 and the hub 14 are advanced. In some aspects or embodiments, the telescoping segments are formed from metal or a plastic.

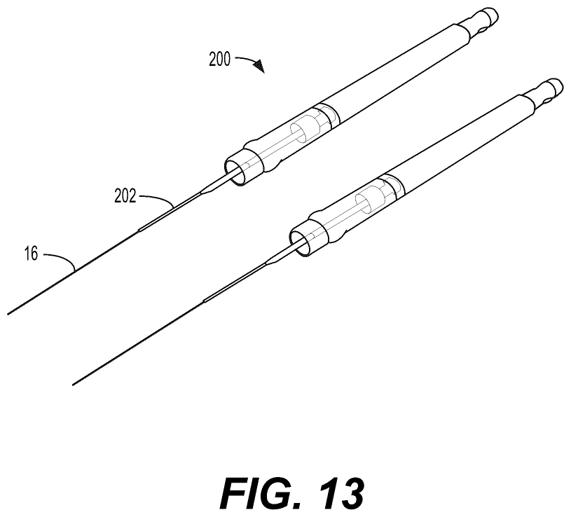

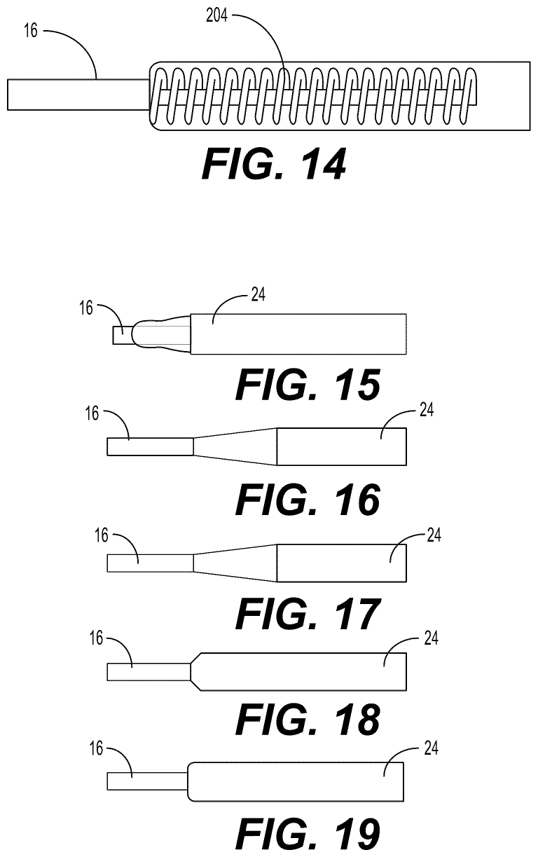

Referring to FIGS. 13 and 14, according to one aspect or embodiment, a line draw device 200 similar to the line draw device 10 is shown. The line draw device 200, however, includes a flexible support tube 202. In some aspects or embodiments, the support tube 202 is formed from semi-rigid, but flexible materials. In some aspects or embodiments, the support tube 202 is formed from polyimide, polyurethane, Nitinol, or combinations thereof. In some aspects or embodiments, the support tube 202 is a polymeric tubing sleeve or a tapered flow tube 16. The flexible support tube 202 is more flexible than a thin wall metal tube, such as the support tube 24 discussed above. As shown in FIG. 14, in one aspect or embodiment, the support tube 202 includes a steel wire coil 204. The steel wire coil 204 may have constant or viable winding. In one aspect or embodiment, the steel wire coil 204 includes a tight wind on a proximal length with a loose wind on a distal end to provide the flow tube 16 with support while being flexible to support the flow tube 16 through angle changes during advancement of the flow tube 16. The steel wire coil 204 adds flexibility during advancement of the flow tube 16, but also can be compressed if a corner or catch point is encountered, to allow for continued advancement of the flow tube 16 despite a distal end of the steel wire coil 204 not continuing to advance.

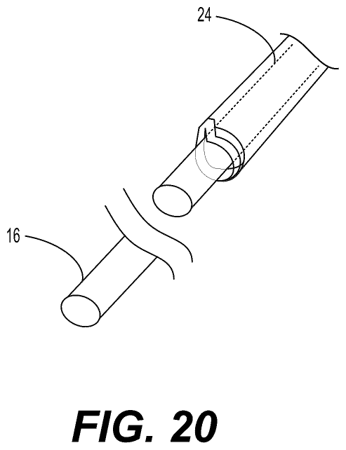

Referring to FIGS. 15-20, according to aspects or embodiments, various concepts for a transition between the support tube 24 and the flow tube 16 that is configured to reduce damage to the indwelling catheter or related components is shown. In some aspects or embodiments, the transition between the support tube 24 and the flow tube 16 includes an overmold (FIG. 15), a tapered segment or portion (FIGS. 16 and 17), a chamfer (FIG. 18), and/or radius (FIG. 19). As shown in FIG. 20, in one aspect or embodiment, the transition between the support tube 24 and the flow tube 16 includes an asymmetrical tip of the support tube 24.

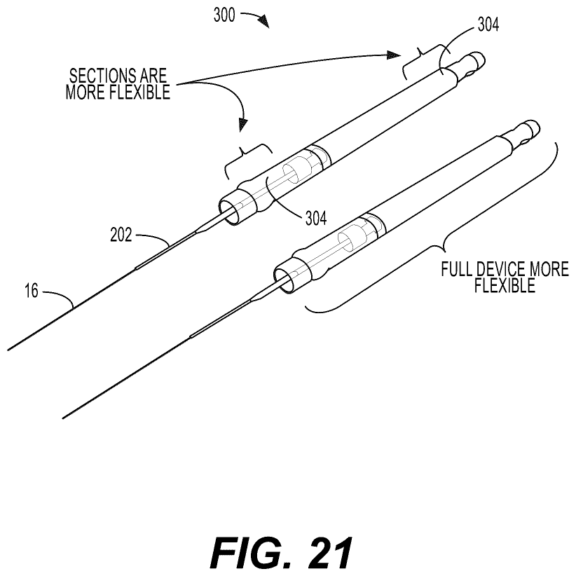

Referring to FIG. 21, according to one aspect or embodiment, a line draw device 300 similar to the line draw device 10 is shown. The line draw device 300, however, includes a flexible housing 302. In some aspects or embodiments, the flexible housing 302 is flexible due to the material and/or shape of the flexible housing 302. In some aspects or embodiments, the flexible housing 302 includes one or more flexible sections 304 rather than or in addition to the entire flexible housing 302 being flexible.

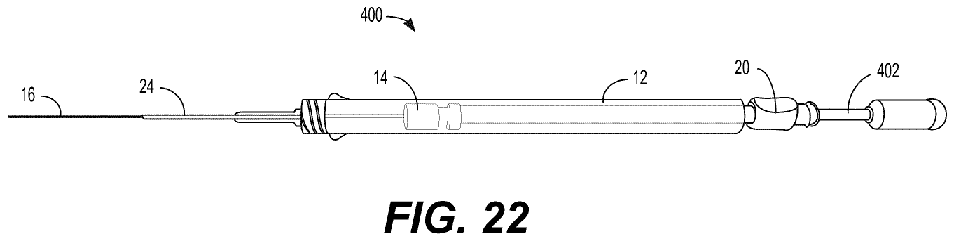

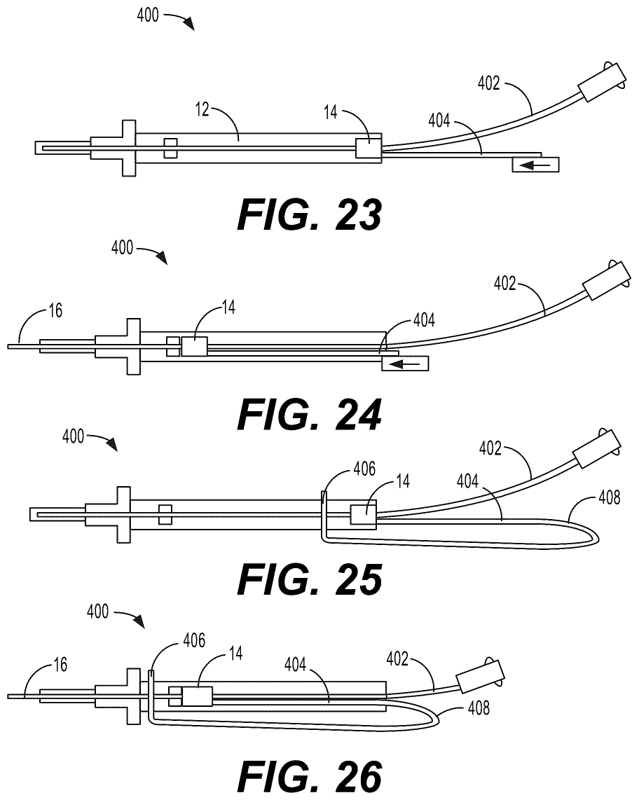

Referring to FIGS. 22-26, according to one aspect or embodiment, a line draw device 400 similar to the line draw device 10 is shown. The line draw device 400, however, includes an extension tube 402 connected to the advancement handle 20 (FIG. 22) or connected to the hub 14 (FIGS. 23-26). As shown in FIGS. 23-26, in some aspects or embodiments, rather than providing the advancement tube 18 as an actuator, the line draw device 400 includes an advancement member 404 as an actuator to move the flow tube 16 between the retracted and extended positions. The extension tube 402 can be aligned with a longitudinal axis of the housing 12 of the device 400 or can be angled or non-parallel to the longitudinal axis of the housing 12. As shown in FIGS. 23 and 24, the extension tube 402 remains flexible to ensure optimal blood draw procedures as the advancement member 404 is moved from a retracted position (FIG. 23) to an advanced position (FIG. 24). As shown in FIGS. 25 and 26, in one aspect or embodiment, the advancement member 404 includes a handle portion 406 configured to move along the housing 12 as the flow tube 16 is moved between the retracted position and the extended position, with the advancement member 404 extending proximally from the hub 14 and extending distally after a transition portion 408.

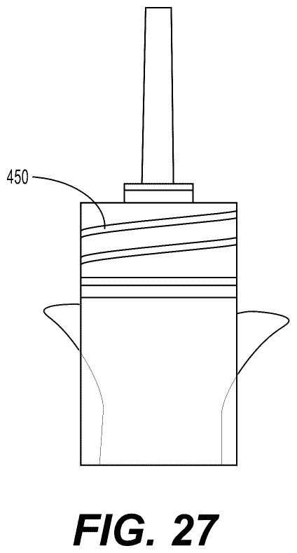

Referring to FIG. 27, in one aspect or embodiment, a connector 450 similar to the connector 22 of the line draw device 10 includes one or more features to inhibit the disconnection of a straight catheter from a needlefree connector as the line draw device 10 is attached when a rotating nut is gripped while attaching the line draw device 10. These features may include: adjusting the Luer Lok thread pitch angle, as shown in FIG. 27; adding friction bumps or other friction creating features to either the needlefree connector spinning nut or the blunt cannula luer of the line draw device 10; changing materials to have a differentiated attachment torque; application of silicone to the blunt cannula and/or the threads of the connector 22; and/or reducing the height or outer diameter of the luer to reduce the compression of the needlefree connector.

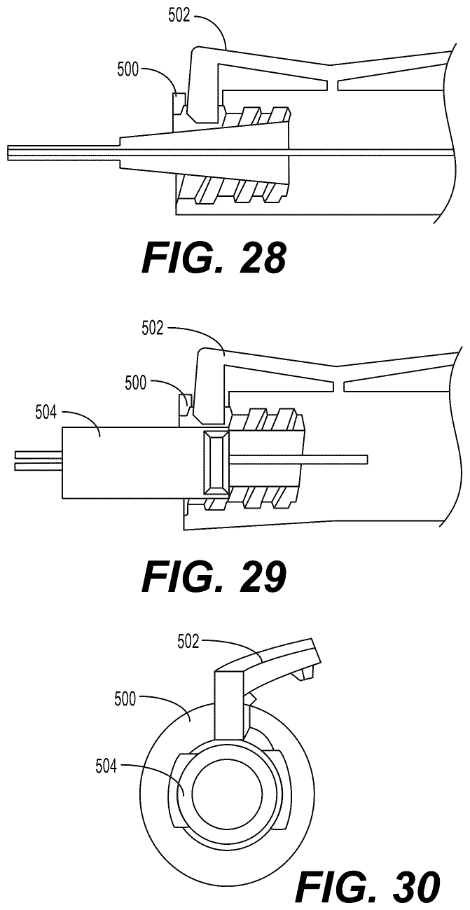

Referring to FIGS. 28-30, in one aspect or embodiment, a connector 500 similar to the connector 22 of the line draw device 10 further includes a moveable lock member 502. When a corresponding component 504, such as a female luer, is received by the male luer lock, the moveable lock member 502 is configured to engage the corresponding component 504 to lock the corresponding component 504 to the housing 12. The moveable lock member 502 is configured to prevent the connector 500 from inadvertently disconnecting from a needlefree or other mating component during use. The moveable lock member 502 is a lever on the outside of the housing 12, with a portion passing through a hole to engage the threads of the corresponding component 504.

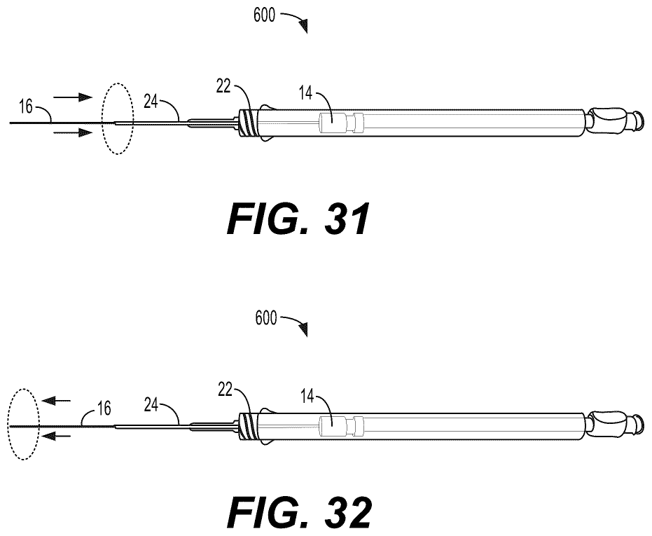

Referring to FIGS. 31 and 32, in one aspect or embodiment, a line draw device 600 similar to the line draw device 10 is shown. The line draw device 600, however, may include one or more features to increase the extension or advancement length of the flow tube 16. Approximately 30 mm of extension of the flow tube 16 beyond the connector 22 is advantageous to achieve optimal blood draw results. In one aspect or embodiment, these features include one or more of the following: overall length of the device increased to add increased stroke; a support 24 that limits the stroke could be shortened or made flexible; and the support 24 could be moveably attached to the flow tube 16 so that when it is at an extended position, as advancement continues, the support 24 could slide along the flow tube 16 allowing further advancement of the flow tube 16 distally relative to the support 24. In one aspect or embodiment, the line draw device 10 is modified such that the metal support tube 24 does not extend beyond a distal end of the connector 22 when the flow tube 16 is in the extended position and when a distal end of the flow tube 16 has a maximum extension from the distal end of the connector 22.

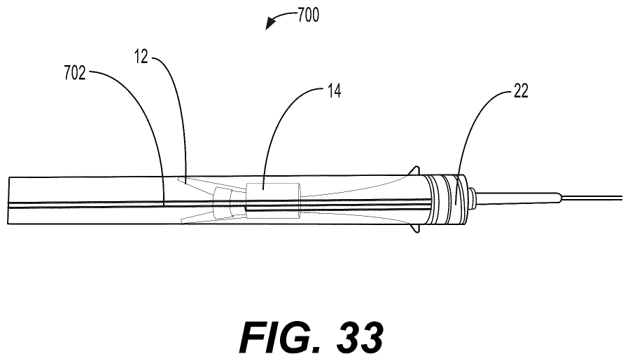

Referring to FIG. 33, in one aspect or embodiment, a line draw device 700 similar to the line draw device 10 is shown. The line draw device 700, however, includes one or more features to prevent or inhibit the premature or inadvertent retraction of the flow tube 16 during use. In one aspect or embodiment, these features include one or more of: a rotational or other motion to enable a retraction block, friction increasing zone, or retraction block; widening or adjustment of a track within the housing 12 to include a single step, multiple steps or an angle to increase the force required to retract the flow tube 16; and a friction zone with increased tightening or other features which increase friction, such as friction bumps. As shown in FIG. 33, the housing 12 includes a track 702 with the hub 14 having a guide (not shown) received within the track 702, with the track including a retraction inhibition feature. In one aspect or embodiment, the retraction inhibition feature includes track structure configured to increase a force required to move the flow tube 16 from the extended position to the retracted position, such as widened or narrowed portions, steps, detent bumps, or other structure. In one aspect or embodiment, the retraction inhibition feature includes non-linear track portions, such as portions that require rotation during advancement of the flow tube 16.

In one aspect or embodiment, a method of using any of the line draw devices 100, 200, 300, 400, 600, 700 includes: connecting a line draw device to an indwelling catheter; advancing the flow tube 16 beyond the tip of the indwelling catheter while limiting the support tube or other feature from advancing beyond the proximal side of any flexible portion of the fluid path and any portion of the fluid path with a centerline or feature angle change; collecting a blood sample; and removing the line draw device from the indwelling catheter.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A line draw device for use with a peripheral intravenous catheter (PIVC), the line draw device comprising:
a housing having a proximal end, a distal end, and a sidewall therebetween defining an inner volume;
a flow tube defining a lumen, the flow tube having a retracted position where a distal end of the flow tube is received within the housing and an extended position where the distal end of the flow tube extends beyond the distal end of the housing;
a support tube connected to the flow tube and moveable with the flow tube; and
a collapsible support positioned within the housing, a portion of the flow tube extending through the collapsible support, wherein the collapsible support has a first length when the flow tube is in the retracted position and a second length when the flow tube is in the extended position, the first length larger than the second length, and wherein a distal end of the support tube is configured to be positioned proximally of a distal end of a needlefree connector when the line draw device is connected to the needlefree connector.

2. The line draw device of claim 1, wherein the collapsible support comprises an accordion fold.

3. The line draw device of claim 1, wherein the collapsible support comprises telescoping segments.

4. A line draw device for use with a peripheral intravenous catheter (PIVC), the line draw device comprising:
a housing having a proximal end, a distal end, and a sidewall therebetween defining an inner volume;
a hub received within the housing;
a flow tube connected to the hub and defining a lumen, the flow tube having a retracted position where a distal end of the flow tube is received within the housing and an extended position where the distal end of the flow tube extends beyond the distal end of the housing; and
a lock member, the lock member configured to engage the hub or housing when the flow tube is in the extended position.

5. A line draw device for use with a peripheral intravenous catheter (PIVC), the line draw device comprising:
a housing having a proximal end, a distal end, and a sidewall therebetween defining an inner volume;
a flow tube defining a lumen, the flow tube having a retracted position where a distal end of the flow tube is received within the housing and an extended position where the distal end of the flow tube extends beyond the distal end of the housing; and
a support tube connected to the flow tube and moveable with the flow tube when the flow tube moves between the retracted position and the extended position, wherein the support tube is flexible.

6. The line draw device of claim 5, wherein the support tube comprises polyimide, polyurethane, Nitinol, or a semi-rigid material more flexible than steel.

7. The line draw device of claim 5, wherein the support tube comprises a steel wire coil.

8. A line draw device for use with a peripheral intravenous catheter (PIVC), the line draw device comprising:
a housing having a proximal end, a distal end, and a sidewall therebetween defining an inner volume;
a flow tube defining a lumen, the flow tube having a retracted position where a distal end of the flow tube is received within the housing and an extended position where the distal end of the flow tube extends beyond the distal end of the housing; and
a support tube connected to the flow tube and moveable with the flow tube when the flow tube moves between the retracted position and the extended position, wherein a transition between the support tube and the flow tube is configured to reduce damage to the PIVC or related components and/or kinking of the flow tube.

9. The line draw device of claim 8, wherein the transition between the support tube and the flow tube comprises an overmold, a tapered segment, a chamfer, and/or radius.

10. The line draw device of claim 8, wherein the transition between the support tube and the flow tube comprises an asymmetrical tip of the support tube.

11. A line draw device for use with a peripheral intravenous catheter (PIVC), the line draw device comprising:
a housing having a proximal end, a distal end, and a sidewall therebetween defining an inner volume;
a flow tube defining a lumen, the flow tube having a retracted position where a distal end of the flow tube is received within the housing and an extended position where the distal end of the flow tube extends beyond the distal end of the housing; and
a support tube connected to the flow tube and moveable with the flow tube when the flow tube moves between the retracted position and the extended position, wherein the housing is flexible.

12. The line draw device of claim 11, wherein the housing comprises one or more flexible sections.

13. A line draw device for use with a peripheral intravenous catheter (PIVC), the line draw device comprising:
a housing having a proximal end, a distal end, and a sidewall therebetween defining an inner volume;
a hub received within the housing;
a flow tube connected to the hub and defining a lumen, the flow tube having a retracted position where a distal end of the flow tube is received within the housing and an extended position where the distal end of the flow tube extends beyond the distal end of the housing;
an actuator connected to the hub, at least a portion of the actuator extends from the proximal end of the housing; and
an extension tube connected to the actuator or the hub.

14. A line draw device for use with a peripheral intravenous catheter (PIVC), the line draw device comprising:
a housing having a proximal end, a distal end, and a sidewall therebetween defining an inner volume;
a hub received within the housing;
a flow tube connected to the hub and defining a lumen, the flow tube having a retracted position where a distal end of the flow tube is received within the housing and an extended position where the distal end of the flow tube extends beyond the distal end of the housing; and
a connector positioned at the distal end of the housing, the connector comprising a male luer lock connector and a moveable lock member, wherein, when a corresponding component is received by the male luer lock connector, the moveable lock member configured to engage the corresponding component to lock the corresponding component to the housing.

15. A line draw device for use with a peripheral intravenous catheter (PIVC), the line draw device comprising:
a housing having a proximal end, a distal end, and a sidewall therebetween defining an inner volume;
a connector positioned at the distal end of the housing;
a flow tube defining a lumen, the flow tube having a retracted position where a distal end of the flow tube is received within the housing and an extended position where the distal end of the flow tube extends beyond the distal end of the housing; and
a support tube connected to the flow tube and moveable with the flow tube when the flow tube moves between the retracted position and the extended position, wherein a distal end of the support tube is positioned proximally of a distal end of the connector when the flow tube is in the extended position and when a distal end of the flow tube has a maximum extension from the distal end of the connector.

16. A system comprising:
an integrated catheter comprising a catheter adapter with a side port and needlefree connector in fluid communication with the side port; and
a line draw device comprising:
a housing having a proximal end, a distal end, and a sidewall therebetween defining an inner volume;
a connector positioned at the distal end of the housing, the connector configured to be connected to the needlefree connector;
a flow tube defining a lumen, the flow tube having a retracted position where a distal end of the flow tube is received within the housing and an extended position where the distal end of the flow tube extends beyond the distal end of the housing; and
a support tube connected to the flow tube and moveable with the flow tube when the flow tube moves between the retracted position and the extended position, wherein a distal end of the support tube is positioned proximally of an internal wall of the catheter adapter when the flow tube is in the extended position and when a distal end of the flow tube has a maximum extension from the distal end of the connector.

17. The system of claim 16, wherein the distal end of the support tube is positioned at a centerline of the catheter adapter when the flow tube is in the extended position and when the distal end of the flow tube has a maximum extension from the distal end of the connector.

18. A line draw device for use with a peripheral intravenous catheter (PIVC), the line draw device comprising:
a housing having a proximal end, a distal end, and a sidewall therebetween defining an inner volume, the housing defining a track;
a hub received within the housing, the hub comprising a guide received within the track of the housing; and
a flow tube connected to the hub and defining a lumen, the flow tube having a retracted position where a distal end of the flow tube is received within the housing and an extended position where the distal end of the flow tube extends beyond the distal end of the housing, wherein the track comprises a retraction inhibition feature.

19. The line draw device of claim 18, wherein the retraction inhibition feature comprises a track structure configured to increase a force required to move the flow tube from the extended position to the retracted position.

20. The line draw device of claim 18, wherein the retraction inhibition feature comprises non-linear track portions.
